(19) United States
(12) Patent Application Publication
(10) Pub. No.: US 2018/0199921 A1
(43) Pub. Date: Jul. 19, 2018

(54) Cell-Based Artificial Olfactory Detector

(71) Applicants: David R. Hall, Provo, UT (US); Terrece Pearman, Draper, UT (US)

(72) Inventors: David R. Hall, Provo, UT (US); Terrece Pearman, Draper, UT (US)

(21) Appl. No.: 15/410,405

(22) Filed: Jan. 19, 2017

(51) Int. Cl. A61B 10/00; C12Q 1/68; G01N 33/52; G01N 33/53

(52) U.S. Cl. CPC A61B 10/00; C12Q 1/6897; G01N 33/521; A61B 2010/0083; A61B 10/0038; A61B 10/007; A61B 2010/0061; G01N 33/5302

(57) ABSTRACT

We disclose a cell-based olfactory sensing device which may be used to diagnose diseases that a mammal, insect, or other organism may diagnose by smell. The device includes a multi-well cell culture plate which cultivates a plurality of cell lines. The cell lines each express a heterologously expressed olfactory receptor gene and a reporter gene. When a ligand from a biological sample derived from a user binds one or more of the olfactory receptors expressed by the cell lines, the reporter genes are expressed. The product of the reporter genes is detectable. Furthermore, the device may be part of a medical toilet. The device may include a controller which may include a memory and a machine-readable medium for analyzing data the device collects.

### BACKGROUND

### Field of the Invention

This disclosure relates to devices and methods for diagnosing disease.

### Background of the Invention

Many animals have heightened senses relative to humans. In fact, humans have used the relatively enhanced ability to see, hear, and smell of animals to perform tasks for hundreds of years. In particular, dogs have been used for their enhanced sense of smell to assist in tasks that include hunting, protecting livestock from predators, searching for specific humans, and detecting illegal substances. More recently, evidence has been reported that dogs have predicted seizures before they happened and identified cancer. Other organisms, including rats, mice, and insects show behavior that suggests they can identify a diseased organism.

It is unclear what substances animals smell when they identify disease. Furthermore, studies show that a variety of biological substances collected from a diseased human emit substances that animals distinguish from those of healthy humans. Reports of animals identifying disease include those in which the animal evaluated feces, urine, blood, and exhaled breath. Each of these biological substances emit volatile organic compounds (VOCs). It is likely that the biological samples the animals identify as those from a diseased human emit a plurality of different VOCs. It may be this combination that the animal perceives as the scent of disease. By smelling the combination of molecules that collectively identify disease, the animal may be able to diagnose with more sensitivity and specificity than available laboratory assays.

In recent years, genes for olfactory receptors in organisms that can identify disease by smell have been cloned and sequenced. This provides a tool to exploit in replicating the olfactory sensation the animal perceives when diagnosing disease without the use of the whole animal. An apparatus and mechanism for diagnosing disease which mimics the olfactory system of animals is needed.

### BRIEF SUMMARY OF THE INVENTION

We disclose a cell-based olfactory sensing device which may diagnose diseases that a mammal, insect, or other organism can diagnose by smell. The device includes a multi-well cell culture plate. The wells within the multi-well cell culture plate may house a plurality of cell lines, with a single cell line in a well. Each of the plurality of cell lines may express a heterologously expressed olfactory receptor and a reporter gene. When VOCs emitted by a biological sample collected from a user are exposed to the plurality of cell lines, those VOCs which are ligands for any of the heterologously expressed olfactory receptors in the cell lines bind the heterologously expressed olfactory receptors. Intracellular signaling mechanisms occur and activate a promoter which regulates expression of a reporter gene. The reporter gene codes for a protein that is directly or indirectly detectable.

The device may include a detector which senses the presence of the protein expressed by the reporter gene. The device also may include a controller which includes a memory for storing data sets. The controller may include a machine-readable medium which may analyze the data sets, provide a diagnosis, and provide an indicator of reliability of the diagnosis.

The device may be included in a medical toilet in which a user may deposit a biological sample. The medical toilet may include an air moving apparatus which directs air into the device. The air may include VOCs emitted by the biological sample.

### BRIEF DESCRIPTION OF THE DRAWINGS

FIG. 1A is a top-down view of an embodiment of a multi-well cell culture plate according to the disclosure.

FIG. 1B is a side view of the multi-well cell culture plate of FIG. 1A.

FIG. 1C is a side view of an embodiment of a multi-well cell culture plate according to the disclosure.

FIG. 2 is a schematic diagram of a heterologous olfactory receptor gene and a reporter gene according to an embodiment of the disclosure.

FIG. 3 is a schematic diagram of a heterologous olfactory receptor gene and a reporter gene in plasm ids that may be transferred into a parent cell line and expressed therein according to an embodiment of the disclosure.

FIG. 4 is a schematic drawing of a cell expressing a heterologous olfactory receptor gene and a reporter gene according to an embodiment of the disclosure.

FIG. 5 is a schematic drawing of a spectrometer detecting a fluorescent signal from a multi-well cell culture plate according to an embodiment of the disclosure.

FIG. 6A is an embodiment of a multi-well cell culture plate according to an embodiment of the disclosure that has been exposed to VOCs from a biological sample obtained from a user in need of a health assessment.

FIG. 6B is an embodiment of a multi-well cell culture plate according to an embodiment of the disclosure that is a positive control plate.

FIG. 6C is an embodiment of a multi-well cell culture plate according to an embodiment of the disclosure that is a negative control plate.

FIG. 7 is a drawing of a medical toilet which includes an embodiment of the disclosed cell-based olfactory sensing device.

FIG. 8 is a flow chart describing steps that may be used when a user provides a biological sample to be used for diagnosis by an embodiment of the disclosed cell-based olfactory sensing device.

### DETAILED DESCRIPTION OF THE INVENTION

Definitions

Toilet, as used herein, means a device that collects biological products of a mammal, including urine, feces, and vomit.

Medical toilet, as used herein, means a toilet that conducts one or more measurements relevant to a user's health status.

User, as used herein, means any mammal, human or animal, for which the cell-based olfactory sensing device disclosed herein is used to provide a health status assessment.

Biological sample, as used herein, means any one or combination of urine, feces, vomit, sputum, blood, seminal fluid, tears, nasal mucus, gastrointestinal tract mucus, urogenital tract mucus, saliva, exhaled breath, or sweat from the body of a user.

Disease, as used herein, means any disorder of structure or function in the body or a human or animal, whether or not the disorder presents with signs or symptoms.

Diseases that may be diagnosed according to the methods disclosed herein and using the cell-based olfactory sensing device disclosed herein include, but are not limited to, colon adenoma, colon carcinoma, colon adenocarcinoma, colorectal adenoma, colorectal carcinoma, colorectal adenocarcinoma, bladder carcinoma, bladder adenocarcinoma, liver adenoma, liver carcinoma, liver adenocarcinoma, esophageal adenoma, esophageal carcinoma, esophageal adenocarcinoma, stomach adenoma, stomach carcinoma, stomach adenocarcinoma, pancreatic adenoma, pancreatic carcinoma, pancreatic adenocarcinoma, lung cancer, mouth cancer, throat cancer, inflammatory bowel disease, urinary tract infection, gastric ulcer, diabetes, hyperglycemia, hypoglycemia, impending seizure, and impending migraine.

While this invention is susceptible of embodiment in many different forms, there are shown in the drawings, which will herein be described in detail, several specific embodiments with the understanding that the present disclosure is to be considered as an exemplification of the principals of the invention and is not intended to limit the invention to the illustrated embodiments.

Disclosed herein is a medical device used to diagnose disease in a user. The device mimics the ability of an animal, insect, or other organism use olfactory sensation to differentiate biological samples collected from a user who suffers from a disease from a user who does not suffer from the disease. In other words, the device mimics the ability of an animal, insect, or other organism to diagnose disease by smell.

The device may include a multi-well cell culture plate. Each well of the multi-well cell culture plate may house a cell line that has been engineered to express two heterologous genes. These heterologous genes include a heterologously expressed olfactory receptor gene and a reporter gene. The parent cell line may be one that is devoid of expression of an endogenous olfactory receptor. Consequently, the heterologous olfactory receptor is the only olfactory receptor the cell line expresses. Different wells in the multi-well cell culture plate may express a different heterologously expressed olfactory receptor. Consequently, a plurality of cultured cell lines may be cultivated in the multi-well cell culture plate.

The cultured cell lines may be created from a single parent cell line. Consequently, the only difference between the different cultured cell lines may be the heterologously expressed genes described herein. The parent cell line may be derived from tissue collected from mammalian or insect tissue or from tissue collected from any other organism which is capable of diagnosing disease by smell.

Expression of the heterologously expressed olfactory receptor gene may be controlled by a heterologous promoter. The heterologous promoter may be a constitutive eukaryotic promoter. The constitutive eukaryotic promoter may be a CMV, TRE, SV40, CAGG, PGK, UBC, or EF1A promoter. Other constitutive eukaryotic promoters known in the art may also be used to control expression of the heterologously expressed olfactory receptor gene.

The heterologously expressed olfactory receptor gene may also include a cDNA that codes of an olfactory receptor (hereinafter, “olfactory receptor cDNA”) positioned downstream of the heterologous promoter such that the heterologous promoter controls expression of the olfactory receptor cDNA. The olfactory receptor cDNA may code for an olfactory receptor derived from a mammal, an insect, or other organism that can diagnose disease based on smell. Each of the plurality of cell lines may express a heterologously expressed olfactory receptor gene that includes a different olfactory receptor cDNA. Consequently, the different cell lines express a different olfactory receptor and detect the presence of a different VOC ligand. When cultured in a multi-well cell culture plate, the plate functions as an array that detects different VOCs similar to the mechanism that olfactory tissue uses in vivo.

In some embodiments, a polyadenylation (polyA+) region may be positioned downstream of the olfactory receptor cDNA. The polyA+ region may provide stability to the mRNA, assist in termination of transcription of the mRNA and in translation, and participate in exportation of the mRNA from the nucleus. Other embodiments may exclude the polyA+ region.

The heterologously expressed olfactory receptor gene may be inserted into a circular plasmid using recombinant DNA techniques known in the art. The circular plasmid may include a gene that expresses a protein that selects for cells that include the circular plasmid. These may include ampicillin resistance gene, tetracycline resistance gene, or other selection mechanisms known in the art.

Like the heterologously expressed olfactory receptor gene, the reporter gene may include its own heterologous promoter, with an activity that is mediated by second messengers and intracellular signaling within the cell line. The intracellular signaling may be initiated when the heterologously expressed olfactory receptor in the cell is bound by its VOC ligand. The intracellular signaling may include increased intracellular levels of second messengers including cyclic adenosine monophosphate (cAMP) or calcium ions.

The second messenger-mediated promoter in the reporter gene may control the expression of a reporter gene cDNA which codes for a reporter protein. The reporter protein may be either directly or indirectly detectable. For example, the reporter protein may be detectable by fluorescent, colorimetric, or other means known in the art. The reporter gene cDNA may be expressed when intracellular signaling activates the second messenger-mediated promoter in the reporter gene. In some embodiments, a polyadenylation (polyA+) region may be positioned downstream of the reporter gene cDNA. Furthermore, the reporter gene may be inserted into a circular plasmid using recombinant DNA techniques known in the art. The circular plasmid may include a gene that expresses a protein that selects for cells that include the circular plasmid. These may include ampicillin resistance gene, tetracycline resistance gene, or other selection mechanisms known in the art.

The plurality of cell lines may be cultured in a suitable growth medium. The growth medium may include the two selection compounds which accomplish the double selection and which encourages survival of cell lines which include both plasmids described herein. In addition to traditional liquid media, the cell lines may be grown on or within protein matrices that comprise cell matrix proteins.

The multi-well cell culture plate may include a port through which VOCs may travel to contact the cell lines within the wells of the multi-well cell culture plate. The port may be in connection with a channel which leads to an airspace surrounding a biological sample collected from a user.

In some embodiments, the port includes a one-way valve. The one-way valve may allow VOCs to enter the multi-well cell culture plate. The one-way valve may also prevent VOCs from exiting the multi-well cell culture plate while the device is in use.

The VOCs may travel through the channel and then through the port where they may contact the cell lines. At this point, they may bind with the heterologously expressed olfactory receptors to which they are ligands. The ligand-bound heterologously expressed olfactory receptors may initiate intracellular signaling events which results in expression of the reporter gene. A detector may sense the protein product of the reporter gene. The detector may transmit the measurements of the protein product of the reporter gene to a controller that includes a memory. The measurements may be stored in the memory.

The controller may also include machine-readable medium which may compare the pattern of reporter gene expression detected in multi-well cell culture plate after exposure to VOCs to patterns detected on multi-well cell culture plates that have been exposed to VOCs collected from individuals known to suffer from specific diseases. The latter patterns may serve as positive controls and aid in diagnosing the user with the disease represented by the patterns.

The machine-readable medium may compare the pattern of reporter gene expression detected in multi-well cell culture plate after exposure to VOCs to patterns detected on multi-well cell culture plates that have been exposed to VOCs collected from health individuals. These patterns may serve as negative controls.

The machine-readable medium may perform a statistical analysis of the comparisons between the pattern of reporter gene expression detected in a multi-well cell culture plate after exposure to VOCs from a user's biological sample with the positive and negative control patterns. This statistical analysis may provide a level of reliability of a diagnosis provided by the disclosed cell-based olfactory sensing device.

In some embodiments, the cell-based olfactory sensing device may be included in a medical toilet. The toilet may include a toilet bowl into which a user may deposit biological samples in the same manner as a traditional toilet. The channel in the cell-based olfactory sensing device may be in connection with the toilet bowl. A fan or other air-moving apparatus known in the art may direct air across the biological sample and into the channel. VOCs emitted from the biological sample may travel with the air into the channel, through a port, and into the multi-well cell culture plate which house the plurality of cell lines disclosed herein.

In some embodiments, the multi-well cell culture plate is housed within a cassette. The cassette may be used to reversibly attach the multi-well cell culture plate to another device. In some embodiments, the cassette may enable the multi-well cell culture plate to be reversibly attached to a detector. In other embodiments, the cassette may enable the multi-well cell culture plate to be reversibly attached to a medical toilet.

The disclosed cell-based olfactory sensing device may be used to diagnose any disease that an animal, insect, or other organism may diagnose by smell. These diseases include cancer. In addition, these diseases include, but are not limited to, colon adenoma, colon carcinoma, colon adenocarcinoma, colorectal adenoma, colorectal carcinoma, colorectal adenocarcinoma, bladder carcinoma, bladder adenocarcinoma, liver adenoma, liver carcinoma, liver adenocarcinoma, esophageal adenoma, esophageal carcinoma, esophageal adenocarcinoma, stomach adenoma, stomach carcinoma, stomach adenocarcinoma, pancreatic adenoma, pancreatic carcinoma, pancreatic adenocarcinoma, lung cancer, mouth cancer, throat cancer, inflammatory bowel disease, urinary tract infection, gastric ulcer, diabetes, hyperglycemia, hypoglycemia, impending seizure, and impending migraine.

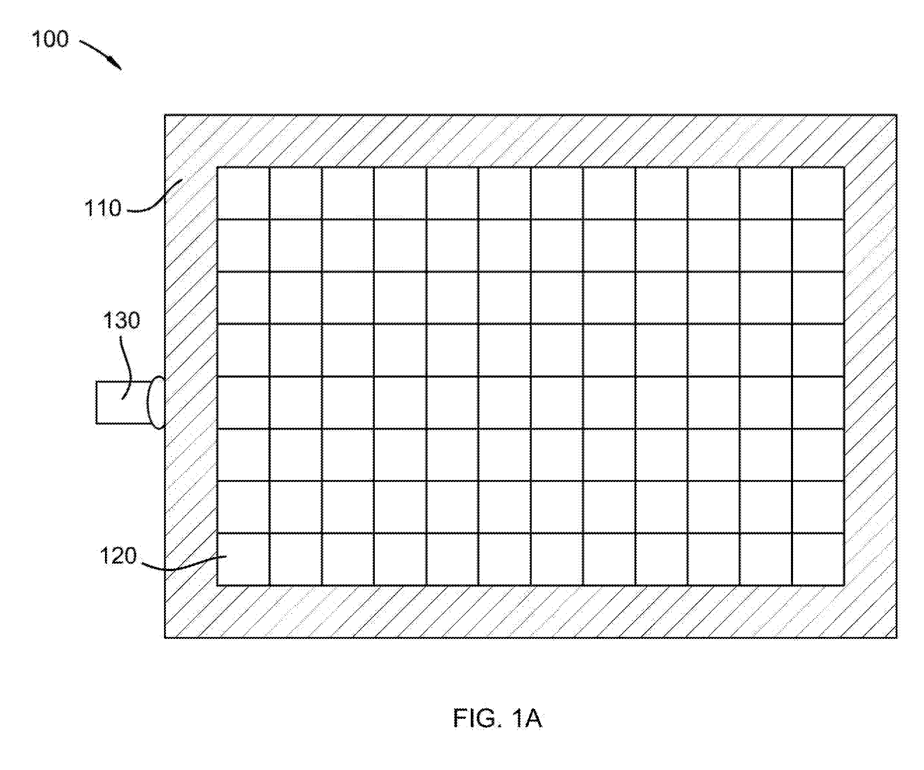

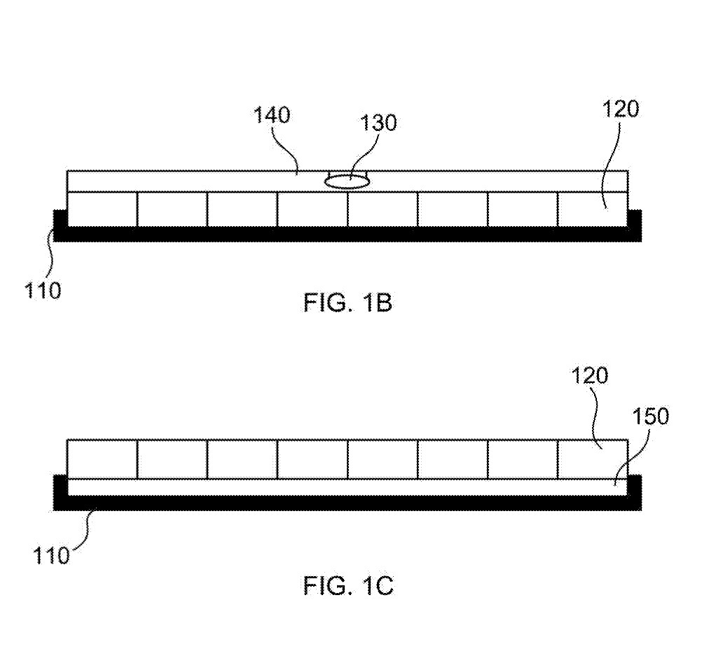

Referring now to the drawings, FIG. 1A illustrates multi-well cell culture plate 100 (hereinafter, “plate 100”) which is an embodiment of the disclosed invention. Plate 100 includes 96 separate wells for culturing cells. Well 120 is one of the 96 separate wells. In some embodiments, the multi-well cell culture plate may include a different number of wells. For example, some multi-well cell culture plates may include 192 wells, 396 wells, 795 wells, 1188 wells, or any number of wells that includes the number of cell cultures desired.

In the embodiment of FIG. 1A, frame 110 surrounds the 96 separate wells. Port 130 may be connected to a section of tubing or other channel or conduit which may transport VOCs emitted by a biological sample into plate 100. The VOCs may travel through port 130 into plate 100 where well 120 and other wells are exposed to the VOCs.

FIG. 1B is a side view of plate 100 shown in FIG. 1A. Port 130 is shown providing an entry for VOCs to enter a space above the wells of plate 100. In this embodiment, the VOCs travel over the wells. The cultured cells absorb the VOCs as they pass above the cultured cells.

FIG. 1C is a side view of another embodiment of a multi-well cell culture plate in which differs from the embodiment of FIGS. 1A and 1B at least because it includes space 150 below the wells housing the cultured cells. This embodiment may include a port or other method of transmitting VOCs into space 150. The multi-well cell culture plate may include a membrane below the wells which is permeable to VOCs but which is impermeable to cells and any growth medium included in the wells. The VOCs may travel through the membrane and contact the cells in the wells.

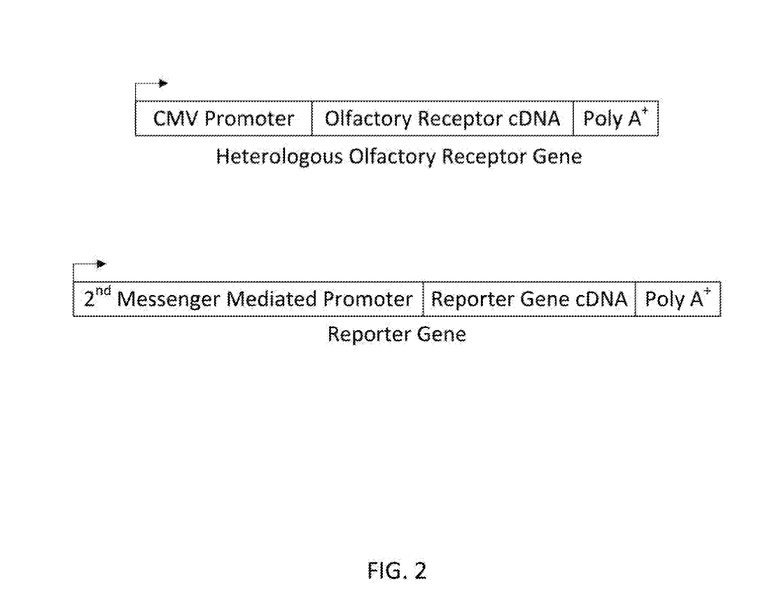

FIG. 2 illustrates embodiments of the genes which may be expressed in the cell lines in the multi-well cell culture plate according to the disclosure. These genes may be constructed using recombinant DNA techniques known in the art. The embodiment of the heterologous olfactory receptor gene shown in FIG. 2 includes a cytomegalovirus (CMV) promoter. This is a constitutive eukaryotic promoter. Other constitutive promoters, including the TRE, SV40, CAGG, PGK, UBC, and EF1A may also be used to drive expression of the heterologous olfactory receptor gene. Promoters that are derived from the same species as the cell line may also be used. Inducible promoters may also be used, although an inducing agent will be added to cause the heterologous olfactory receptor gene to express its product.

FIG. 2 shows an olfactory receptor cDNA downstream of the CMV promoter in the heterologous olfactory receptor gene. The olfactory receptor cDNA may be derived from a mammal, an insect, or any species which is able to use olfactory tissue to differentiate between VOCs from a healthy user and a user with a disease. The olfactory receptor cDNA may be created using any tissue that expresses the olfactory receptor. This may include, but is not limited to, tissue derived from mammalian olfactory sensory neurons, airway epithelial cells, skin, liver, heart, kidneys, sperm, testes, insect antenna and other insect sensory organs. Different wells within the multi-well cell culture plate may include cells that express a different olfactory receptor cDNA.

The heterologous olfactory receptor gene may also include a section that codes for a polyadenylation (polyA+) region. The RNA that is produced from the heterologous olfactory receptor gene according to the embodiment of FIG. 2, will include a polynucleotide chain that includes continuous adenine molecules. Embodiments of the heterologous olfactory receptor gene which do not include a polyA+ region are also within the scope of this disclosure.

FIG. 2 further illustrates an embodiment of a reporter gene. In this embodiment, the reporter gene includes a second messenger-mediated promoter which initiates reporter gene expression in response to a second messenger and other intracellular signaling within the cell. The second messenger may be cyclic AMP (cAMP) or calcium ions. The second messenger-medicated promoter may be activated by a second messenger that is released in response to the olfactory receptor being bound by its ligand.

The reporter gene may further include a report gene cDNA. The reporter gene cDNA may encode any protein that is directly or indirectly detectable and not otherwise expressed in the cell line. Examples include, but are not limited to, green fluorescent protein (GFP), blue fluorescent protein (BFP), red fluorescent protein, luciferase, beta-galactosidase, and chloramphenicol acetyltransferase. In some embodiments, the reporter gene cDNA may be derived from an organism other than which the cell line is derived. Like the heterologous olfactory receptor gene, the reporter gene may also include a region coding for a polyA+ region.

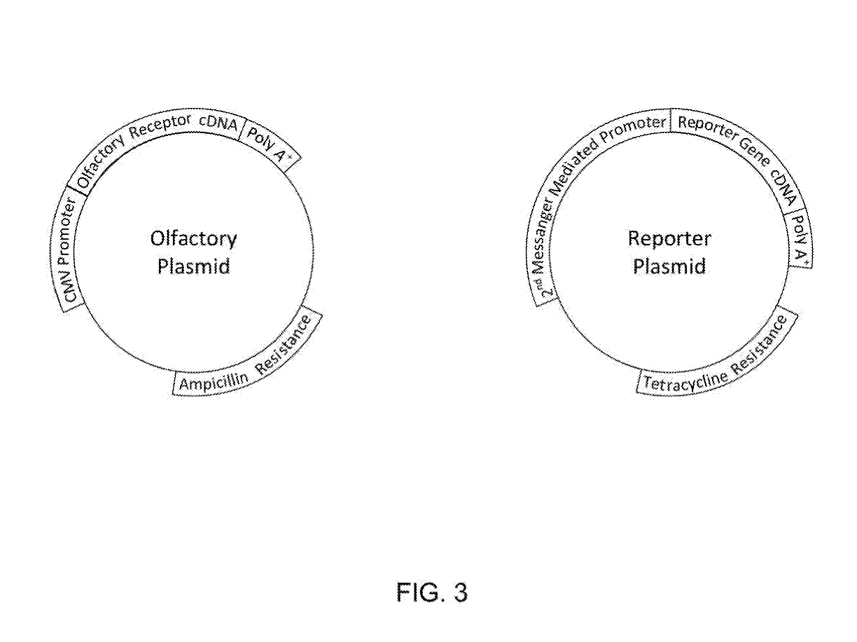

FIG. 3 illustrates embodiments of a heterologous olfactory receptor gene and reporter gene cloned into expression plasm ids. The heterologous olfactory receptor gene is shown in a plasmid that includes an ampicillin resistance gene. Any cell that expresses the heterologous olfactory receptor gene plasmid will be resistant to ampicillin. The reporter gene is shown cloned into a plasmid that includes a tetracycline resistance gene. Any cell that expresses the reporter gene plasmid will be resistant to tetracycline. Both plasmids may be selected for by including both ampicillin and tetracycline in the growth media for the cell lines. Other selection methods known in the art may also be used to perform the double selection.

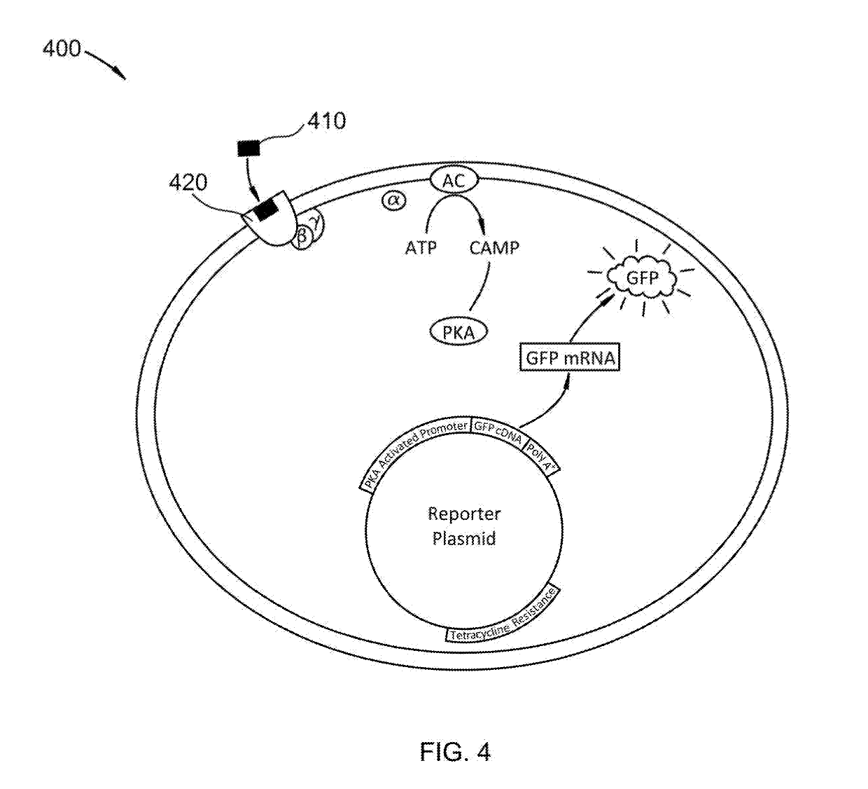

FIG. 4 illustrates cell 400 into which both an embodiment of the heterologous olfactory receptor gene plasmid and the reporter gene plasmid have been introduced. The drawing includes a schematic illustration of olfactory receptor 420. In this embodiment, olfactory receptor 420 has been expressed by an embodiment of a heterologous olfactory receptor gene which has been inserted into a plasmid similar to that shown in FIG. 3. VOC 410 is a ligand for olfactory receptor 420. Upon binding of VOC 410 to olfactory receptor 420, an alpha (α) subunit of a G-protein associated with olfactory receptor 420 separates from a beta (β) and a gamma (γ) subunit of the G-protein. The gamma (γ) subunit activates adenylate cyclase (AC) in the cell membrane. AC converts adenosine triphosphate (ATP) to cyclic adenosine monophosphate (cAMP). In this embodiment, cAMP activates protein kinase A (PKA) which then activates proteins that activates the PKA activated promoter in the reporter gene plasmid within the cell. The reporter gene cDNA in this embodiment is green fluorescent protein (GFP) cDNA. Upon activation of the PKA activated promoter, the GFP cDNA is transcribed to GFP mRNA. The GFP mRNA is then translated to GFP protein. A detector may then detect a fluorescent signal emitted by the GFP protein.

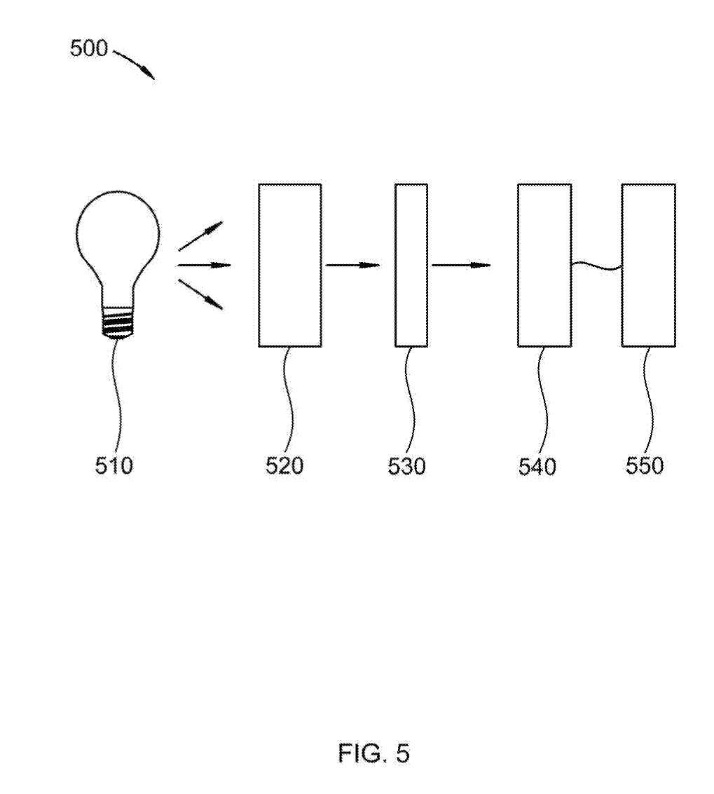

FIG. 5 illustrates detecting system 500 in which a signal emitted by cells in multi-well cell culture plate 530 is being detected by spectrometer 540. Light source 510 emits light (shown as multiple arrows) that comprises multiple wavelengths. The light travels through filter 520 which filters out all but one or a range of wavelengths of light (shown as a single arrow). The filtered light passes through the wells of multi-well cell culture plate 530. Spectrometer 540 measures emitted light of a defined wavelength or range of wavelengths. The emitted light measurement is stored in a memory within controller 550. Controller 550 may also include a machine-readable medium which performs comparisons of the pattern of reporter gene expression by the cells in multi-well cell culture plate 530 with positive and negative controls.

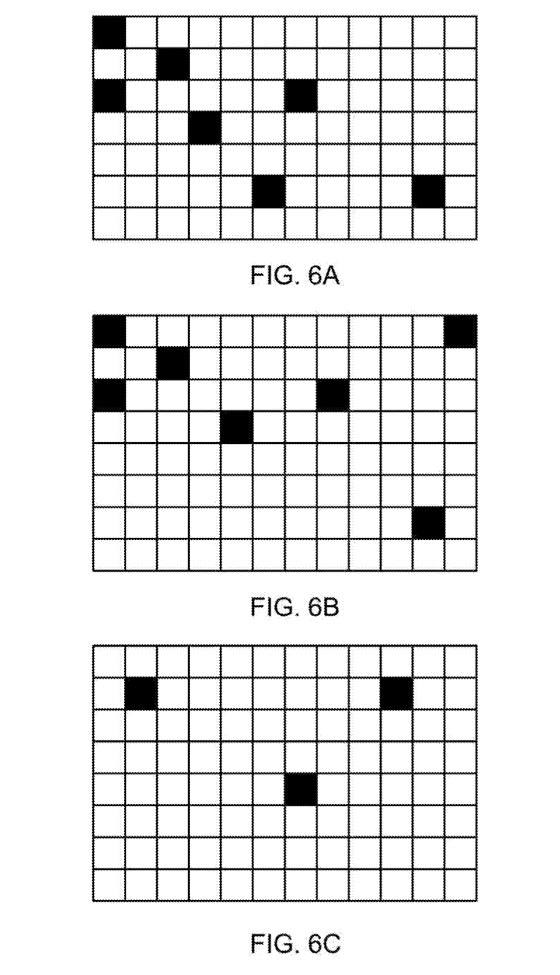

FIG. 6A illustrates an embodiment of a multi-well cell culture plate. This multi-well cell culture plate has been exposed to VOCs emitted by a biological sample which was collected from a user who suffers from a disease. In this embodiment, the disease is one that is detectable by sensing the pattern of VOCs emitted by the biological sample and for which the olfactory receptor expression pattern is known. The darkened wells in the multi-well cell culture plate indicate those wells which house cell lines that express an olfactory receptor which binds one of the VOCs emitted by the user's biological sample. The pattern of olfactory receptors that are bound by the VOCs in the user's biological sample may be compared to a positive control and a negative control plate pattern as shown in FIGS. 6B and 6C.

FIG. 6B illustrates an embodiment of a multi-well cell culture plate which is a positive control plate for the disease discussed in the explanation of FIG. 6A above. The darkened wells in the multi-well cell culture plate indicate those wells which house cell lines that express an olfactory receptor which binds one of the VOCs emitted by a biological sample collected from an individual known to suffer from the disease the user of the multi-well cell culture plate of FIG. 6A is suspected of having. While the pattern of the multi-well cell culture plate of FIG. 6B is not identical to that of FIG. 6A, a machine-readable medium may perform a statistical analysis of the comparison to determine whether the patterns are close enough to provide a reliable diagnosis.

FIG. 6C illustrates an embodiment of a multi-well cell culture plate which is a negative control. The darkened wells in the multi-well cell culture plate indicate those wells which house cell lines that express an olfactory receptor which binds one of the VOCs emitted by a biological sample collected from healthy individual. More specifically, the pattern is that which occurs when the user does not suffer from the disease the user of the multi-well cell culture plate of FIG. 6A is suspected of having.

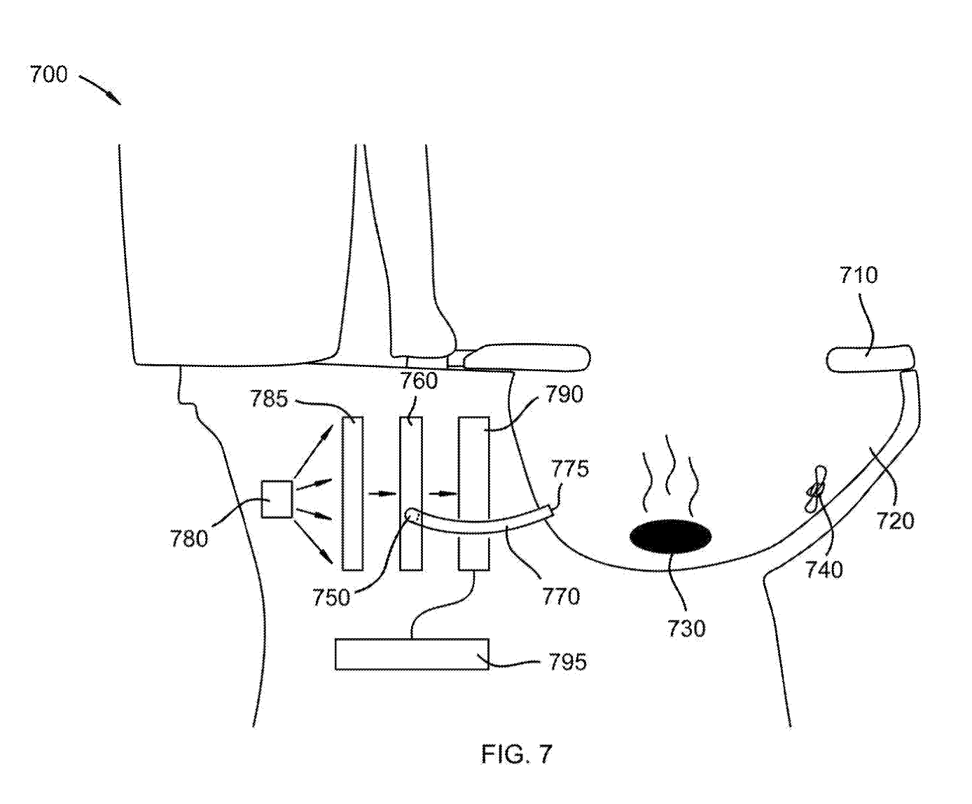

FIG. 7 illustrates medical toilet 700 which includes the disclosed cell-based olfactory sensing device. Medical toilet 700 includes seat 710 and toilet bowl 720. A user has deposited biological sample 730 which emits VOCs as illustrated by the wavy lines. Fan 740 moves air across toilet bowl 720. VOCs emitted from biological sample 730 move with the air towards channel 770. The VOCs travel into channel 770 through orifice 775. The VOCs then enter multi-well cell culture plate 760 through port 750.

Medical toilet 700 includes a detecting system that is similar to the embodiment of FIG. 5. Light source 780 emits light of multiple wavelengths as shown by the multiple arrows. The light travels through filter 785. The filtered light is either a single wavelength or a range of wavelengths as illustrated by the single arrow. The filtered light passes through the wells of multi-well cell culture plate 760 and the light emitted from the wells of multi-well cell culture plate 760 is detected by spectrometer 790. The measurements collected by spectrometer 790 are stored and analyzed in controller 795.

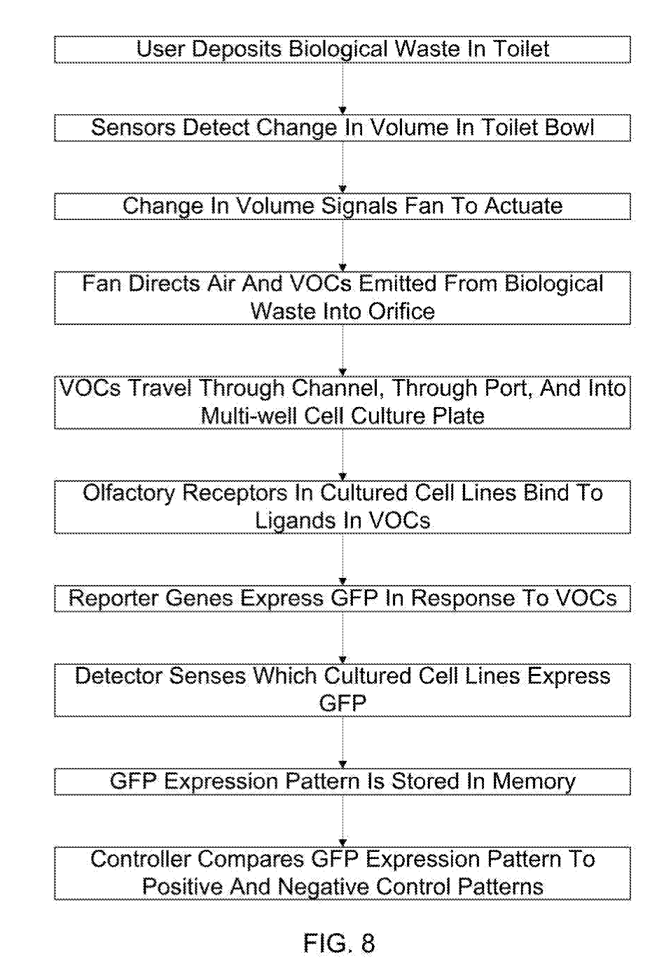

FIG. 8 illustrates a flow chart which includes steps that may be performed during use of the disclosed cell-based olfactory sensing device shown in FIG. 7. In this embodiment, a user deposits a biological sample into a medical toilet that includes the disclosed cell-based olfactory sensing device. The biological sample may be urine, feces, vomit, or other biological material. Sensors in the medical toilet detect a change in volume in the toilet bowl which indicates that a biological sample has been deposited in the toilet bowl. The change in volume sends a signal to actuate a fan within the toilet bowl. The fan directs air across the toilet bowl taking VOCs emitted by the biological sample with the air. The air and VOCs travel into an orifice connected to a channel. The channel leads to a port which is in communication with the interior of a multi-well cell culture plate. The wells of the multi-well cell culture plate house cells lines that express both a heterologous olfactory receptor gene and a reporter gene. The VOCs, which are ligands to the heterologous olfactory receptors, bind their respective olfactory receptors. Cell signaling occurs which induces receptor gene expression. In this embodiment, the reporter gene expresses GFP. A detector measures which cell lines express GFP and the GFP expression pattern in the multi-well cell culture plate is stored in a memory within a controller. The controller compares the pattern of GFP expression on the multi-well cell culture plate with positive and negative control patterns as illustrated by FIGS. 6B and 6C respectively.

While specific embodiments have been illustrated and described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

We claim:

1. A cell-based olfactory sensing device comprising:
a plurality of cultured cell lines, wherein each of the plurality of cultured cell lines comprises:
a heterologously expressed olfactory receptor, wherein the heterologously expressed olfactory receptor is the only type of olfactory receptor the plurality of cultured cell lines express, and wherein the heterologously expressed olfactory receptor is expressed by a heterologous olfactory receptor gene, the heterologous olfactory receptor gene comprising:
a first heterologous promoter, and
a first coding region comprising an olfactory receptor cDNA, wherein the olfactory receptor cDNA codes for the heterologously expressed olfactory receptor, and wherein the olfactory receptor cDNA is expressed under the regulation of the first heterologous promoter;
a reporter gene, wherein the reporter gene comprises:
a second heterologous promoter, wherein the second heterologous promoter is responsive to intracellular signaling that is initiated upon activation of the heterologously expressed olfactory receptor, and
a reporter gene cDNA, wherein the reporter gene cDNA codes for a detectable reporter protein, and wherein expression of the reporter gene cDNA is induced upon activation of the second heterologous promoter,
wherein each of the plurality of cultured cell lines expresses a different heterologously expressed olfactory receptor;
a multi-well cell culture plate comprising a plurality of wells, wherein each of the plurality of wells comprises no more than one of the plurality of cultured cell lines; and
a detector, wherein the detector scans each of the plurality of wells of the multi-well cell culture plate, and wherein the detector senses the presence of the reporter protein; and
a medical toilet, wherein the medical toilet comprises:
a toilet bowl;
a channel connecting the toilet bowl with the multi-well cell culture plate; and
a fan, wherein the fan directs air from the toilet bowl into the channel.

2. The cell-based olfactory sensing device of claim 1, wherein the first coding region codes for an olfactory receptor found in either a mammal or an insect.

3. The cell-based olfactory sensing device of claim 1, wherein plurality of cultured cell lines are created from a single parent cell line.

4. The cell-based olfactory sensing device of claim 3, wherein the single parent cell line is derived from either mammalian or insect tissue.

5. The cell-based olfactory sensing device of claim 1, wherein the reporter protein emits a fluorescent signal.

6. The cell-based olfactory sensing device of claim 1, wherein the multi-well cell culture plate further comprises a port, wherein the port is in fluid communication with the plurality of cultured cell lines.

7. The cell-based olfactory sensing device of claim 1, wherein the multi-well cell culture plate is within a cassette.

8. The cell-based olfactory sensing device of claim 7, wherein the cassette is reversibly connected to the medical toilet.

9. The cell-based olfactory sensing device of claim 1, further comprising a controller, wherein the controller comprises a memory for storing a pattern of expression of the reporter protein in the plurality of cultured cell lines.

10. The cell-based olfactory sensing device of claim 9, wherein the controller further comprises a machine-readable medium, wherein the machine-readable medium compares the pattern of expression of the reporter protein in the plurality of cultured cell lines with a positive control pattern, the positive control pattern resulting from exposing the cell-based olfactory sensing device to volatile organic compounds derived from a biological sample obtained from an individual suffering from a disease, and with a negative control pattern, the negative control pattern resulting from exposing the cell-based olfactory sensing device to volatile organic compounds derived from a biological sample obtained from a healthy individual.

11. The cell-based olfactory sensing device of claim 10, wherein the disease is selected from one or more of the following:
colon adenoma, colon carcinoma, colon adenocarcinoma, colorectal adenoma, colorectal carcinoma, colorectal adenocarcinoma, bladder carcinoma, bladder adenocarcinoma, liver adenoma, liver carcinoma, liver adenocarcinoma, esophageal adenoma, esophageal carcinoma, esophageal adenocarcinoma, stomach adenoma, stomach carcinoma, stomach adenocarcinoma, pancreatic adenoma, pancreatic carcinoma, pancreatic adenocarcinoma, lung cancer, mouth cancer, throat cancer, inflammatory bowel disease, urinary tract infection, gastric ulcer, diabetes, hyperglycemia, hypoglycemia, impending seizure, and impending migraine.

12. The cell-based olfactory sensing device of claim 10, wherein the disease consists of cancer.

13. The cell-based olfactory sensing device of claim 10, wherein the machine-readable medium calculates a statistical analysis of the similarity of the pattern of expression of the reporter protein in the plurality of cultured cell lines with the positive control pattern and with the negative control pattern.

14. A cell-based olfactory sensing device comprising:
a plurality of cultured cell lines, wherein each of the plurality of cultured cell lines comprises:
a heterologously expressed olfactory receptor, wherein the heterologously expressed olfactory receptor is the only type of olfactory receptor the plurality of cultured cell lines express, and wherein the heterologously expressed olfactory receptor is expressed by a heterologous olfactory receptor gene, the heterologous olfactory receptor gene comprising:
a first heterologous promoter, and
a first coding region comprising an olfactory receptor cDNA, wherein the olfactory receptor cDNA codes for the heterologously expressed olfactory receptor, and wherein the olfactory receptor cDNA is expressed under the regulation of the first heterologous promoter;
a reporter gene, wherein the reporter gene comprises:
a second heterologous promoter, wherein the second heterologous promoter is responsive to intracellular signaling that is initiated upon activation of the heterologously expressed olfactory receptor, and
a reporter gene cDNA, wherein the reporter gene cDNA codes for a detectable reporter protein, and wherein expression of the reporter gene cDNA is induced upon activation of the second heterologous promoter,
wherein each of the plurality of cultured cell lines expresses a different heterologously expressed olfactory receptor;
a multi-well cell culture plate comprising a plurality of wells, wherein each of the plurality of wells comprises no more than one of the plurality of cultured cell lines; and
a detector, wherein the detector scans each of the plurality of wells of the multi-well cell culture plate, and wherein the detector senses the presence of the reporter protein.

15. The cell-based olfactory sensing device of claim 14, wherein the first coding region codes for an olfactory receptor found in either a mammal or an insect.

16. The cell-based olfactory sensing device of claim 14, wherein plurality of cultured cell lines are created from a single parent cell line.

17. The cell-based olfactory sensing device of claim 16, wherein the single parent cell line is derived from either mammalian or insect tissue.

18. The cell-based olfactory sensing device of claim 14, wherein the reporter protein emits a fluorescent signal.

19. The cell-based olfactory sensing device of claim 14, wherein the multi-well cell culture plate further comprises a port, wherein the port is in fluid communication with the plurality of cultured cell lines.

20. The cell-based olfactory sensing device of claim 14, wherein the multi-well cell culture plate is within a cassette.
